(19) United States
(12) Patent Application Publication
(10) Pub. No.: US 2024/0060094 A1
(43) Pub. Date: Feb. 22, 2024

(54) Improvements In Biomass Fermentation into Ethanol

(71) Applicant: SixRing Inc., Calgary (CA)

(72) Inventors: Alexandra OSTASZEWSKI, Calgary (CA); Julie GREER, Calgary (CA); Alejandra ENRIQUEZ, Calgary (CA); Kyle G. WYNNYK, Calgary (CA); Andrew CORBETT, Calgary (CA); Markus WEISSENBERGER, Calgary (CA)

(21) Appl. No.: 18/452,169

(22) Filed: Aug. 18, 2023

(30) Foreign Application Priority Data
Aug. 19, 2022 (CA) 3170772

(51) Int. Cl. C12P 7/10

(52) U.S. Cl. CPC C12P 7/10

(57) ABSTRACT

A method of obtaining ethanol from a lignocellulosic biomass comprises delignification of a lignocellulosic biomass using a modified Caro's acid. Next a solid portion of the delignification reaction mixture is recovered wherein the solid portion comprises a substantially hemicellulose-free cellulosic component which includes at most, up to 15% w/w hemicellulose. Next, the recovered solid portion of the resulting reaction mixture is exposed to an enzyme mix comprising cellulase enzymes to create a saccharification system that breaks down the cellulose into a saccharified composition such as oligosaccharides. The saccharified composition is then fed to an organism, such as yeast, with the ability to ferment sugars into ethanol.

### FIELD OF THE INVENTION

The present invention is directed to the use of a cellulose which is free or substantially-free of hemicellulose in the generation of bioethanol.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for bioethanol production is starch which can yield its sugar much more readily than cellulose. This is due to the difference in structure as starch links glucose molecules together through α-1,4 linkages and cellulose links glucose with β-1,4 linkages. The β-1,4 linkages allow for crystallization of the cellulose, leading to a more rigid structure which is more difficult to break down.

The limitation that comes from solely concentrating the bioethanol on extracting the sugars from starches prevents the utilization of the larger portion of biomass which comes in the form of lignocellulosic biomass (contains lignin, cellulose, and hemicellulose) present in almost every plant on earth. A delignification reaction allows the recovery of cellulose from those lignocellulosic plants. Once the cellulose is separated from the other two biomass constituents i.e., lignin, and hemicellulose, further degradation of the cellulose generates cellobiose and/or glucose which can be further processed to bio-ethanol.

Seen as a sustainable alternative to gasoline and with the goal of alleviating many countries' dependence on foreign oil, the bioethanol industry is still hampered by its dependence on corn or sugar cane as their main sources of fuel, as they are both rich in starch. It is estimated that about 45% of all corn production in the U.S. is directed to the ethanol fuel production. This is a situation which has disastrous consequences when the prices of gasoline go so low as to make corn-based bioethanol unsustainable on a price viewpoint.

Across the world, many other large ethanol-producing countries, including China and Brazil, have shown some struggles in ethanol production from biomass as many companies are carrying large debts from the implementation of such processes and large plants have had to shut down or decrease production.

In Asia, palm oil prices have recently increased to their highest levels in years, which, in turn, will hamper the ability of Indonesia and Malaysia to produce local biofuel. Oil palm trunk contains a large amount of starch which is more readily solubilized in water, compared to cellulose. Starch can then be heated and hydrolyzed to glucose by amylolytic enzymes without pre-treatment. However, the conventional Oil palm trunk treatment requires high capital and operational costs and is therefore prohibitive to market entry. Moreover, the treatment carries a high probability of microbial contamination during starch processing.

In Europe, the biofuel industry (both biodiesel and bioethanol production) depends heavily on food-based feedstocks like virgin vegetable oils (i.e., rapeseed, palm oil, soy) for biodiesel and corn, wheat, and sugar beet for bioethanol. Simultaneously, concerns have been raised that making fuel out of crops displaces other crops and can inflate food prices. These concerns are leading to policy changes that incentivize a shift away from food-based biofuels.

To pivot from starches to cellulose for the production of glucose is preferable as it will provide near-unlimited amount of feedstock from waste biomass and reduce the competition with food source feedstock to generate glucose. However, the costs to do so are currently prohibitive. Cellulosic ethanol as it is called relies on the non-food part of a plant to be used to generate ethanol. This would allow the replacement of the current more widespread approach of making bioethanol by using corn or sugarcane. The diversity and abundance of these types of cellulose-rich plants would allow to maintain food resources mostly intact and capitalize on the waste generated from these food resources (such as cornstalk) to generate ethanol. Other cellulose sources such as straws, algae and even trees fall under the cellulose-rich biomass which can be used in generating ethanol if a commercially viable process is developed.

The reason why starches are preferred to cellulose-rich sources to generate ethanol is that extraction of glucose from cellulose is substantially more difficult and resource intensive. To better understand the difference which raises this difficulty it is worthwhile pointing the similarities and differences between starch and cellulose.

Cellulose and starch are polymers which have the same repeat units of glucose. However, the differences between starch and cellulose can be seen in way the repeating glucose monomers are connected to one another. In starch, the glucose monomers are oriented in the same direction. In cellulose, each successive glucose monomer is rotated 180 degrees in respect of the previous glucose monomer. This, in turn, ensures that the bonds between each monomeric glucose differs between starch and cellulose. In starch, the bonds (otherwise known as links) are referred to as α-1,4 linkages, in cellulose these bonds are referred to as β-1,4 linkages.

The difference between these bonds impacts the characteristics of starch and cellulose. Starch can dissolve in warm water while cellulose does not. Starch can be digested by humans, cellulose cannot. Starch is weaker than cellulose partly due to the fact that its structure is less crystalline than cellulose. Starch is, at its core, a method for plants to store energy, therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds. Glucose has two isomers: α-glucose (present in starches as branched polymers) and β-glucose (present in cellulose and connected via a β-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed and, in turn, provide the structural strength needed to support plants.

Hydrolysis of Cellulose

The hydrolysis of cellulose to glucose is the rate limiting step in the conversion of cellulose into biofuel. The processes currently using cellulose as a starting material for bioethanol production require the conversion of cellulose into cellobiose, then glucose, prior to the ultimate generation of ethanol. The fermentation of glucose using yeast is what leads to the production of ethanol. The rate limiting step is the most crucial one and one which hinders a wider acceptance of biofuels. The difficulty in overcoming this conversion of cellulose into glucose lies with the fact that cellulose has a crystalline structure which renders its conversion to glucose quite difficult because of the close packing of multiple cellulose polymers. This close packing imparts on cellulose its inherent stability under a variety of chemical conditions. Cellulose polymers are generally insoluble in water, as well as a number of organic solvents. Cellulose is also generally insoluble when exposed to weak acids or bases.

In general, there are two main approaches to hydrolyze cellulose: chemical and enzymatic. The chemical method resorts to the use of concentrated strong acids to hydrolyze cellulose under conditions of high temperature and pressure. Many different types of acids, such as HCl and H₂SO₄, have been used in the past to achieve this. The use of one of these acids usually results in at least one of the following drawbacks: corrosion of the reaction vessel; difficulty of disposing of the discharged reactants; and others. The biofuel industry is generally reticent to use chemically hydrolyzed cellulose because of the presence of toxic by-products in the resulting glucose. These by-products, if introduced in the fermentation step, will negatively affect the delicate balance of the fermenting yeast.

Cost of Enzymatic Hydrolysis

It is known that the costs to extract biofuel from cellulose are higher than when doing so from starch. It is estimated that, on average, depending on location and availability of biomass, the cost for cellulose conversion is about 50% more than the starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases and hemicellulases). The generation of enzymes for enzymatic hydrolysis of cellulose is a time-consuming process and large volumes of enzymes are required to render the process commercially viable. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising endo-1,4-β-glucanases; exo-1,4-β-glucanases; and β-glucosidases) and hemicellulases (for example, β-xylosidase) require extensive controls in place to maximize the reaction rates the enzymatic approach is expected to provide. Temperature, pH, salinity, concentration of substrate and product are all factors that may affect enzyme activity. Small deviations from the enzyme's optimal conditions will result in loss of function. The conversion of cellulose to glucose is done by a few different enzymes: endo-1,4-β-glucanases; exo-1,4-β-glucanases; and β—glucosidases, all of which have specific environmental conditions which must be met. These controls render the process cost prohibitive in some cases and/or limiting in their implementation.

PCT patent application WO9640970 (A1) discloses a method of producing sugars from materials containing cellulose and hemicellulose comprising: mixing the materials with a solution of about 25-90% acid by weight thereby at least partially decrystallizing the materials and forming a gel that includes solid material and a liquid portion; diluting said gel to an acid concentration of from about 20% to about 30% by weight and heating said gel to a temperature between about 80° C. and 100° C. thereby partially hydrolyzing the cellulose and hemicellulose contained in said materials; separating said liquid portion from said solid material, thereby obtaining a first liquid containing sugars and acid; mixing the separated solid material with a solution of about 25-90% acid until the acid concentration of the gel is between about 20-30% acid by weight and heating the mixture to a temperature between about 80° C. and 100° C. further hydrolyzing cellulose and hemicellulose remaining in said separated solid material and forming a second solid material and a second liquid portion; separating said second liquid portion from said second solid material thereby obtaining a second liquid containing sugars and acid; combining the first and second liquids; and separating the sugars from the acid in the combined first and second liquids to produce a third liquid containing a total of at least about 15% sugar by weight, which is not more than 3% acid by weight.

In the paper entitled ‘A novel facile two-step method for producing glucose from cellulose’ (Bioresource Technology Volume 137, June 2013, Pages 106-110) a two-step acid-catalyzed hydrolysis methodology is disclosed where cellulose is hydrolyzed to glucose with high yield and selectivity under mild conditions. Its approach involves a multi-step hydrolysis, comprising as first step, the depolymerization of microcrystalline cellulose in phosphoric acid to cellulose oligomer at 50° C. The second step involves the precipitation of the oligomer by ethanol and subsequent hydrolysis with dilute sulfuric acid.

In the paper entitled ‘Dilute-acid Hydrolysis of Cellulose to Glucose from Sugarcane Bagasse’ from Dussan et al. (CHEMICAL ENGINEERING TRANSACTIONS VOL. 38, 2014), there is disclosed a method of generating ethanol through the hydrolysis of cellulose. Sugarcane bagasse is used as a substrate for ethanol production, optimum conditions for acid hydrolysis of cellulose fraction were assessed. The glucose thus generated was fermented to ethanol using yeast (Scheffersomyces stipitis).

The hydrolysis of cellulose is, as seen from the above, limited by the structure of cellulose itself but also by the approaches taken to degrade to glucose. The production of a robust, low-cost process from cellulose has not yet been achieved.

The benefits of bioethanol are estimated to have the potential to reduce gas emissions by up to 85% over reformulated gasoline. However, numerous production challenges to generate bioethanol from lignocellulosic biomass rather than from starch have led experts in the field to conclude that, in the near future, cellulosic ethanol will not be produced in sufficient quantities to provide at least a partial gasoline replacement or alternative. It is important that second-generation bioethanol production be based on the use of lignocellulosic biomass as a starting material in order to render it environmentally desirable and economically feasible.

However, the microbial fermentation of xylose, which is the main pentose sugar present in hemicellulose, is a limiting factor in developing such processes. Since some current process to remove lignin from lignocellulosic biomass leave a large portion of hemicellulose present with the cellulose, there remains a high concentration of xylose present following enzymatic or chemical hydrolysis of cellulose. The presence of xylose causes substantial difficulties for the fermentation of cellulose to ethanol to produce bioethanol as native species of common fermenters used, for example S. cerevisiae, are not able to ferment both glucose and xylose. As hemicellulose is converted to xylose, this can lead to the formation of organic acids, such as acetic acid, and furaldehydes. Accumulation of these compounds will result in inhibition of yeast metabolism, thus reducing bioethanol production. Some approaches employed to overcome the limitations caused by the presence of hemicellulose have been to genetically modify yeast to ferment both glucose and xylose, however multiple there are multiple genetic hurdles to overcome, as fermentation of xylose will result in xylitol, which will also inhibit the yeast metabolism.

One of the approaches to dealing with the presence of xylose in pulp for conversion into ethanol is to use a mixture of different microorganisms (co-culturing), some of which can convert glucose into ethanol and other which can ferment xylose into xylulose and ultimately into ethanol. Examples of organisms capable of accomplishing the latter conversion route are Pichia stipites (reclassified as Scheffersomyces supitis) and Kluyveromyces marxianus. The main drawback of the use of these strains are the lower ethanol yields, the inability to grow without oxygen as well as the sensitivity to low concentrations of inhibitors in hydrolysates (i.e., acetate).

Another approach involving the use of a strain of the white rot basidiomycete Trametes versicolor that was found to be capable of fermenting xylose was recently developed. It was also shown to be capable of converting non-pretreated starch, cellulose, xylan, wheat bran and rice straw into ethanol and survive the effect of certain common inhibitors. These research and findings are recent and thus require more research to assess if, and how, it can be implemented on a large scale.

Another approach for the conversion of xylose to ethanol involves the use of yeasts. Several yeasts including Saccharomyces cerevisiae and Schizosaccharomyces pombe have been modified to be capable of converting xylose directly into ethanol, however there are several challenges which makes these strains impractical for large scale applications.

Many of the current pulping approaches yield pulp which needs to be bleached to remove the remaining lignin (around 3-4% of the treated biomass) and still contain a non-negligible hemicellulose content (around 20% of the remaining treated biomass). The hemicellulose is the source of the xylose whose presence slows down the fermentation of the cellulose into ethanol.

In light of the state of the art with respect to the use of lignocellulosic biomass in the manufacturing of bioethanol, there is still a need for a process which is capable of being scaled up efficiently which allows the use of lignocellulosic biomass in the manufacturing of bioethanol. Preferably, it is also desirable to overcome the drawbacks associated with the presence of hemicellulose in the pulp which undergoes fermentation to cellulose. The aforementioned is also substantiated given the tremendous efforts to convert waste biomass to biofuels using different approaches which have almost all failed to achieve this goal for subsequent conversion to glucose and ultimately, ethanol.

The inventors have surprisingly and unexpectedly found that the characteristics of the cellulose obtained from a specific type of delignification approach have a substantial impact on the downstream hydrolysis of cellulose into glucose and subsequent conversion into ethanol.

### SUMMARY OF THE INVENTION

Lignocellulosic biomass is a widely available resource which is the feedstock used in second-generation bioethanol production. However, the incomplete removal of hemicellulose during the pre-treatment of cellulose impedes the efficiency of the fermentation process due to, for example, slow xylose transport, inefficient co-utilization of glucose and xylose, inefficient downstream pathway metabolism, the functional expression of xylA and overall lower ethanol production. As such, it is preferable to minimize the amount of hemicellulose remaining in the pulp when the latter is used in the production of bioethanol in order to maximize thereof.

According to an aspect of the present invention, there is provided a method of increasing the efficiency of cellulose fermentation into bioethanol by removing or substantially reducing the amount of hemicellulose present in the biomass. By removing most of the hemicellulose from a lignocellulosic biomass material, the amount of ethanol from pure cellulose (not from the cellulose+hemicellulose mixture) is thus maximized. This leads to lower costs in the production of bioethanol since the fermentation is simpler and does not require genetically modified organisms. A genetically modified yeast would need to be present to ferment the pentose sugars (xylose) when such a cellulose/hemicellulose blend is present, as a standard fermentative yeast will not be as efficient. The presence of hemicellulose/xylose does not benefit the production of bioethanol using a standard yeast, so the overall yields of biomass conversion to bio-ethanol will suffer.

According to an aspect of the present invention, there is provided a method of increasing the fermentation yield of cellulose by first treating the biomass to a delignification reaction and then collecting the remaining solids in large part comprised of cellulose and fermenting said cellulose in an appropriate biodigester wherein said cellulose is fermented into ethanol. Preferably, the absence of hemicellulose in the cellulose resulting in a favorable ethanol yield compared to cellulose containing hemicellulose.

It is to be understood that the presence of a low amount of hemicellulose (0.5 to 15 wt. %) will have generally much improved yields in comparison to conventional cellulose which contains larger percentages (15-25 wt. %) of hemicellulose therein. For instance, since hemicellulose is in general, the second most common constituent of lignocellulosic biomass, it is expected that it be present in a range of 15-25% in a conventional pulp after delignification using the kraft process.

Preferably, the addition of a substantially-free of hemicellulose biomass additive allows for an increase in the generation of ethanol in a fermentation unit when the biomass additive is used as part of the organic waste being fermented or as the entire organic load in the saccharification or fermentation unit. The substantially-free of hemicellulose cellulose will undergo a hydrolysis reaction using chemicals, organisms or enzymes to generate glucose and other oligosaccharides, known as a saccharification reaction. This saccharified component will then undergo a fermentation in order to generate ethanol.

According to an aspect of the present invention, there is provided a use of a cellulosic component comprising cellulose and hemicellulose, said cellulosic component obtained from a delignification process using a modified Caro's acid selected from the group consisting of:
composition A; composition B and Composition C;
wherein said composition A comprises:
   sulfuric acid in an amount ranging from 20 to 70 wt % of the total weight of the composition;
   a modifier component comprising an amine moiety and a sulfonic acid moiety selected from the group consisting of: taurine; taurine derivatives; and taurine-related compounds; and
   a peroxide;
wherein said composition B comprises:
   an alkylsulfonic acid; and
   a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
   sulfuric acid;
   a two-part modifier component comprising:
      a compound comprising an amine moiety;
      a compound comprising a sulfonic acid moiety; and
   a peroxide;
      for the saccharification of cellulose into glucose and subsequent fermentation into ethanol, wherein said cellulosic component comprises at least 85 wt % of said cellulose and, at most, 15 wt % of said hemicellulose as a result of said delignification process.

According to an aspect of the present invention, there is provided a use of a cellulosic component comprising cellulose, hemicellulose and lignin obtained from the treatment of lignocellulosic biomass with a modified Caro's acid in the fermentation of cellulose into cellobiose and ethanol, where the cellulosic component is characterized in that its content of lignin is below 1 w/w % and hemicellulose is below 15 w/w % wherein said modified Caro's acid is selected from the group consisting of: composition A; composition B and Composition C;
wherein said composition A comprises:
   sulfuric acid in an amount ranging from 20 to 70 wt % of the total weight of the composition;
   a modifier component comprising an amine moiety and a sulfonic acid moiety selected from the group consisting of: taurine; taurine derivatives; and taurine-related compounds; and
   a peroxide;
wherein said composition B comprises:
   an alkylsulfonic acid; and
   a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
   sulfuric acid;
   a two-part modifier component comprising:
      a compound comprising an amine moiety;
      a compound comprising a sulfonic acid moiety; and
   a peroxide.

According to a preferred embodiment of the present invention, the cellulose to hemicellulose weight ratio in the cellulosic component is 6:1 or more. Preferably, the cellulose to hemicellulose weight ratio in the cellulosic component is 7:1 or more. Preferably, the cellulose to hemicellulose weight ratio in the cellulosic component is 10:1 or more. More preferably, the cellulose to hemicellulose weight ratio in the cellulosic component is 12:1 or more.

According to an aspect of the present invention, there is provided a use of a substantially hemicellulose-free cellulosic component as an additive to organic material intended for bioethanol production, wherein said use increases the amount of ethanol produced from a fermentation unit.

According to a preferred embodiment of the present invention, the cellulosic component comprises at least 85% cellulose. Preferably, the substantially hemicellulose-free cellulosic component comprises at least 90% cellulose. More preferably, the substantially hemicellulose-free cellulosic component comprises at least 92.5% cellulose.

According to another aspect of the present invention, there is provided a method of obtaining ethanol from a lignocellulosic biomass where said method comprises the following steps:
Step 1: preparing a delignification mixture and delignifying a lignocellulosic biomass using a modified Caro's acid selected from the group consisting of: composition A; composition B and Composition C;
   wherein said composition A comprises:
      sulfuric acid in an amount ranging from 20 to 70 wt % of the total weight of the composition;
      a modifier component comprising an amine moiety and a sulfonic acid moiety selected from the group consisting of: taurine; taurine derivatives; and taurine-related compounds; and
      a peroxide;
   wherein said composition B comprises:
      an alkylsulfonic acid; and
      a peroxide; wherein the acid is present in an amount ranging from 40 to 80 wt % of the total weight of the composition and where the peroxide is present in an amount ranging from 10 to 40 wt % of the total weight of the composition;
   wherein said composition C comprises:
      sulfuric acid;
         a two-part modifier component comprising:
         a compound comprising an amine moiety;
         a compound comprising a sulfonic acid moiety; and
         a peroxide;
            wherein said delignification mixture comprises said lignocellulosic biomass and said modified Caro's acid;
Step 2: recovering a solid portion of said delignification reaction mixture, wherein said solid portion comprises a substantially hemicellulose-free cellulosic component which comprises at most, up to 15 w/w hemicellulose;
Step 3: exposing said recovered solid portion of the resulting reaction mixture to an enzyme mix comprising at least one cellulase enzyme to create a saccharification or fermentation system which breaks down the cellulose into a saccharified composition, e.g., oligosaccharides or ferments said saccharified composition to ethanol;
Step 4: optionally, feeding the saccharified composition to an organism, such as yeast, capable of fermenting sugars into ethanol.

Preferably, the method further comprising a step 5, where, after fermentation, the liquid portion from the fermentation system is distilled to recover said ethanol.

According to another aspect of the present invention, there is provided a method to increase the amount of ethanol produced from a fermentation system by using a substantially hemicellulose-free cellulosic component, which comprises at most, up to 15% w/w hemicellulose, as an additive to organic material intended for bioethanol production. The increase is in comparison to a system which uses a cellulosic component with much higher hemicellulose content such as 20% or more.

According to yet another aspect of the present invention, there is provided a method to increase the amount of ethanol produced from a fermentation system by using a substantially hemicellulose-free cellulosic component, which comprises at most, up to 15% w/w hemicellulose, as a partial replacement to organic material intended for bioethanol production.

Preferably, said substantially hemicellulose-free cellulosic component comprises less than 30% of the amount hemicellulose present prior to a lignocellulosic biomass being delignified. More preferably, said substantially hemicellulose-free cellulosic component comprises less than 20% of the amount hemicellulose present prior to a lignocellulosic biomass being delignified. Even more preferably, the substantially hemicellulose-free cellulosic component comprises less than 8% of the amount hemicellulose present prior to a lignocellulosic biomass being delignified. Yet even more preferably, the substantially hemicellulose-free cellulosic component comprises less than 4% of the amount hemicellulose present prior to a lignocellulosic biomass being delignified.

According to yet another aspect of the present invention, there is provided a process to hydrolyze cellulose into glucose, said process comprising the following steps:
providing a reaction vessel;
providing a source of cellulose into said reaction vessel; wherein said source of cellulose has a hemicellulose content of less than 30% of the original hemicellulose content of lignocellulosic biomass from which it originated;
providing a microbial inoculum into said reaction vessel;
exposing said inoculum to said source of cellulose in an aqueous medium of pH of about 8 at a temperature ranging from 30° C. to 40° C. for a period of time ranging from 1 to 42 days;
optionally, recovering the supernatant comprising glucose; and
exposing the glucose to a bacterium, a fungi, or yeast, or a combination thereof which ferments the glucose to ethanol.

According to yet another aspect of the present invention, there is provided a process to hydrolyze cellulose into glucose, said process comprising the following steps:
providing a reaction vessel;
providing a source of cellulose into said reaction vessel; wherein said source of cellulose has a hemicellulose content of less than 30% of the original hemicellulose content of lignocellulosic biomass which it originated from;
providing an enzyme blend into said reaction vessel;
exposing said enzyme blend to said source of cellulose in an aqueous medium of pH of about 4-6 at a temperature ranging from 40° C. to 55° C. for a period of time ranging from 1 to 7 days;
optionally, recovering the supernatant comprising glucose; and
exposing the glucose to a bacterium, a fungi, a yeast, or a combination thereof which ferments the glucose to ethanol.

The term “saccharification system” refers to a vessel to which a biomass or a cellulosic component is added alongside a chemical, organism, or enzyme blend under such conditions as to convert complex sugars (i.e., polysaccharides including cellulose and hemicellulose) to simple sugars such as oligo, di-, and monosaccharides (i.e., glucose, xylose, etc.).

The term “fermentation system” refers to a vessel to which a mixture of oligo, di-, and monosaccharides (i.e., glucose, xylose, etc.) is added alongside an ethanologenic organisms and kept under such conditions as to convert the sugars into ethanol. In some aspects of this invention, the term “fermentation system” also refers to a vessel in which a biomass or a cellulosic component is added alongside a combination of chemicals, organisms, and/or enzyme blends under such conditions as to convert complex sugars (i.e., polysaccharides including cellulose and hemicellulose) directly to ethanol in a one-pot process.

The term “substantially free of hemicellulose” or “substantially hemicellulose-free” refers to a biomass additive or a cellulosic component comprising less than 15% hemicellulose, preferably less than 10% hemicellulose, more preferably less than 7.5% hemicellulose.

According to a preferred embodiment of the present invention, the biomass additive is cellulose which has been processed to be substantially free of hemicellulose. Preferably, the biomass additive contains at most 30% of the original hemicellulose content from the harvested lignocellulosic biomass. Preferably, the biomass additive contains at most 20% of the original hemicellulose content from the harvested lignocellulosic biomass. Preferably, the biomass additive contains at most 10% of the original hemicellulose content from the harvested lignocellulosic biomass. Preferably, the biomass additive contains at most 8% of the original hemicellulose content from the harvested lignocellulosic biomass. Preferably, the biomass additive contains at most 6% of the original hemicellulose content from the harvested lignocellulosic biomass. Preferably, the biomass additive contains at most 5% of the original hemicellulose content from the harvested lignocellulosic biomass. Preferably, the biomass additive contains at most 4% of the original hemicellulose content from the harvested lignocellulosic biomass. Preferably, the biomass additive contains at most 2% of the original hemicellulose content from the harvested lignocellulosic biomass. Preferably, the biomass additive contains at most 1% of the original hemicellulose content from the harvested lignocellulosic biomass. According to a preferred embodiment of the present invention, the biomass additive labelled as substantially free of hemicellulose also contains at most 1 w/w % of lignin.

When resorting to a cellulose which was delignified using a modified Caro's acid and performed according to a process described herein, the remaining hemicellulose can hover as low as 5% or even less of the total weight of the pulp. Similarly, the hemicellulose is hydrolyzed at the same time and the sugars forming such are solubilized and remain in the liquid phase. After the delignification is deemed sufficiently complete for the purposes of the operator, the solids (cellulose and residual hemicellulose up to 0.5 to 15 w/w %) are separated from the liquid containing the modified Caro's acid as well as lignin fragments and hemicellulose fragments (of which xylose is a constituent). This approach maximizes the hemicellulose removal from the cellulose and allows conventional enzymes or the like to be used to convert the extracted cellulose into ethanol in an efficient manner. This also removes the necessity of finding a mixture of various enzymes capable of converting cellulose and hemicellulose into ethanol, thus streamlining the process and ensuring a more efficient conversion of lignocellulosic biomass into ethanol.

### BRIEF DESCRIPTION OF THE FIGURES

Features and advantages of embodiments of the present application will become apparent from the following detailed description and the appended figures, which:

FIG. 1 is a graphical depiction of the glucose production of the samples as per experiment #1;

FIG. 2 is a graphical depiction of the xylose production of the samples as per experiment #1; and

FIG. 3 is a graphical depiction of the ethanol production of the samples as per experiment #1.

### DETAILED DESCRIPTION OF THE INVENTION

The delignification of biomass according to conventional approaches such a kraft pulping, yields a pulp which is still high in lignin and hemicellulose. By adding a cellulose-rich additive which is essentially devoid of hemicellulose (which hydrolyzes to xylose), it has been made possible to increase the generation of ethanol from the fermentation of glucose.

According to a preferred embodiment of the present invention, the cellulose is an unbleached cellulose which has a very low content in hemicellulose (preferably ranging from 0.5 to 15 w/w %). Preferably, the cellulose is obtained by the delignification of a lignocellulosic biomass feedstock through the exposure of such to a modified Caro's acid as per the following processes. A preferred embodiment of the process to delignify biomass, comprises the steps of:
providing a vessel;
providing biomass comprising lignin, hemicellulose and cellulose fibers into said vessel;
providing a sulfuric acid component;
providing a peroxide component;
exposing said biomass to said sulfuric acid source and peroxide component;
allowing said sulfuric acid source and peroxide component to come into contact with said biomass for a period of time sufficient to a delignification reaction to occur and remove over 90 wt % of said lignin and hemicellulose from said biomass.

Preferably, the biomass comprising lignin, hemicellulose and cellulose fibers is exposed to a modified Caro's acid composition selected from the group consisting of: composition A; composition B and Composition C;
wherein said composition A comprises:
   sulfuric acid in an amount ranging from 20 to 70 wt % of the total weight of the composition;
   a modifier component comprising an amine moiety and a sulfonic acid moiety selected from the group consisting of: taurine; taurine derivatives; and taurine-related compounds; and
   a peroxide;
wherein said composition B comprises:
   an alkylsulfonic acid; and
   a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
   sulfuric acid;
   a two-part modifier component comprising:
      a compound comprising an amine moiety;
      a compound comprising a sulfonic acid moiety; and
   a peroxide.

According to a preferred embodiment of the present invention, substantially hemicellulose-free cellulose means that the cellulosic component comprises at least 85% cellulose. Preferably, the cellulosic component comprises at least 90% cellulose. More preferably, the cellulosic component comprises at least 92.5% cellulose.

Preferably, the delignification reaction is carried out at a temperature below 55° C. by a method selected from the group consisting of:
adding water into said vessel;
adding biomass into said vessel; and
using a heat exchanger.

Preferably, said sulfuric acid, said modifier component comprising an amine moiety and a sulfonic acid moiety and said peroxide are present in a molar ratio of no less than 1:1:1. Also preferably, said sulfuric acid, said compound comprising an amine moiety and a sulfonic acid moiety and said peroxide are present in a molar ratio of no more than 15:1:1.

According to a preferred embodiment of the present invention, said sulfuric acid and said compound comprising an amine moiety and a sulfonic acid moiety are present in a molar ratio of no less than 3:1.

According to a preferred embodiment of the present invention, said compound comprising an amine moiety and a sulfonic acid moiety is selected from the group consisting of: taurine; taurine derivatives; and taurine-related compounds.

According to a preferred embodiment of the present invention, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl. Preferably, said linear alkylaminosulfonic acid is selected form the group consisting of: methyl; ethyl (taurine); propyl; and butyl. Preferably, said branched aminoalkylsulfonic acid is selected from the group consisting of: isopropyl; isobutyl; and isopentyl.

According to a preferred embodiment of the present invention, said compound comprising an amine moiety and a sulfonic acid moiety is taurine.

According to a preferred embodiment of the present invention, said sulfuric acid and compound comprising an amine moiety and a sulfonic acid moiety are present in a molar ratio of no less than 3:1.

According to a preferred embodiment of the present invention, said compound comprising an amine moiety is an alkanolamine is selected from the group consisting of: monoethanolamine; diethanolamine; triethanolamine; and combinations thereof.

According to a preferred embodiment of the present invention, said compound comprising a sulfonic acid moiety is selected from the group consisting of: alkylsulfonic acids and combinations thereof.

According to a preferred embodiment of the present invention, said alkylsulfonic acid is selected from the group consisting of: alkylsulfonic acids where the alkyl groups range from C₁-C₆ and are linear or branched; and combinations thereof.

According to a preferred embodiment of the present invention, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

According to a preferred embodiment of the present invention, said alkylsulfonic acid; and said peroxide are present in a molar ratio of no less than 1:1.

According to a preferred embodiment of the present invention, said compound comprising a sulfonic acid moiety is methanesulfonic acid.

According to a preferred embodiment of the present invention, in Composition C, said sulfuric acid and said a compound comprising an amine moiety and said compound comprising a sulfonic acid moiety are present in a molar ratio of no less than 1:1:1.

According to a preferred embodiment of the present invention, in Composition C, said sulfuric acid, said compound comprising an amine moiety and said compound comprising a sulfonic acid moiety are present in a molar ratio ranging from 28:1:1 to 2:1:1.

Preferably, for a modified Caro's acid comprising sulfuric acid, peroxide and taurine (as the modifier component), the molar composition is as follows: H₂O:H₂O₂:H₂SO₄:Taurine in a molar ratio of 56:10:10:1.

Preferably, for a modified Caro's acid comprising TEOA/MSA, the molar composition is as follows: H₂O:H₂O₂:H₂SO₄:TEOA:MSA in a molar ratio of 56:10:10:1:1.

In kraft pulping, about 90% of the lignin present in the processed biomass is dissolved and removed therefrom. Kraft pulp also contains hemicellulose fragments (containing xylose) which are detrimental to the proper performance of a fermentation unit. In fact, Kraft pulping dissolves only between 0 to 60% of the hemicellulose initially present in the lignocellulosic feedstock. Therefore, it is clear that the present invention overcomes the shortcomings of the state of the art to produce bioethanol on a large scale using lignocellulosic biomass (or feedstock). Moreover, large scale implementation of preferred embodiments of the methods taught herein will allow large scale bioethanol production from lignocellulosic biomass rather than from starches (such as corn).

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

### Experiment #1—Demonstration of the Increase in Xylose Production with Increasing Hemicellulose Concentration

For this experiment, substantially hemicellulose-free cellulosic component was generated from hardwood forestry waste through the use of a delignification process as disclosed herein, which uses a modified Caro's acid delignification process. As chemicals from the delignification process are recycled, different batches of substantially hemicellulose-free cellulose were generated and labeled with the number of times the chemicals have been reused. As the number of recycles increases, the hemicellulose content increases in parallel. The modified Caro's acid blend make-up used in Experiment #1 is found in Table #1 below:

TABLE 1
Composition of modified Caro's acid used in the preparation of the
cellulosic component of experiment #1

| | | Mass w/w % of the |
|---|---|---|
| Component | Molar Ratio | composition |
| H2O | 56 | 38.53 |
| H2O2 | 10 | 14.46 |
| H2SO4 | 10 | 41.69 |
| Taurine | 1 | 5.32 |

Samples of hardwood biomass were delignified as per the method described herein comprising a modified Caro's acid. A mixture comprising 38.53% w/w of water, 14.46% w/w hydrogen peroxide, 42.69% H₂SO₄ and 5.32% w/w of taurine was prepared. To that, said hardwood biomass was added in a 5.0% loading and the reaction was left stirring in a reactor vessel for 20 hours at 37° C. After the reaction was considered complete, the substantially hemicellulose-free cellulose was filtered out from the liquid stream and neutralized to achieve a pH between 5-7.

The cellulosic component makeup of the various batches prepared including hemicellulose content can be seen in Table 2. A substantially hemicellulose-free cellulose was also generated through the same delignification process but followed with an additional caustic treatment (CT) to further remove the hemicellulose. Hemicellulose content of this cellulose batch can be seen in Table 2. By comparison, raw hardwood biomass has a hemicellulose content of 15-25%.

For the caustic treated sample, 3.0 g of said substantially hemicellulose-free cellulose generated through the fresh delignification using a modified Caro's acid described herein (R2B014) was placed in 100 mL of a 8.5% NaOH solution. The reaction mixture was left stirring at room temperature for 30 minutes, after which, the solution was filtered and the solids were collected.

TABLE 2
Cellulose, hemicellulose and lignin content of the cellulosic
component generated from the delignification of hardwood biomass
using a modified Caro's acid

| | Kappa | Lignin | Hemicellulose | Cellulose |
|---|---|---|---|---|
| Blend | number | % | % | % |
| R2B014 | 1.04 | 0.16% | 7.6% | 92.2% |
| R2B014-1 | 1.33 | 0.20% | 8.9% | 90.9% |
| R2B014-2 | 1.93 | 0.29% | 9.7% | 90.0% |
| R2B014-3 | 2.06 | 0.31% | 10.6% | 89.1% |
| R2B014-4 | 4.14 | 0.62% | 11.9% | 87.5% |
| R2B014-CT | <0.5 | <0.08% | 0.71% | 99.3% |

This experiment tested the conversion of different recycle batches of a substantially hemicellulose-free cellulosic component as well as a sample where a caustic treatment was applied post-delignification. Using a commercially available blend of cellulase enzymes, the cellulosic component was loaded at a 5% w/w in buffer, and samples were saccharified using a commercially available enzyme blend at 50° C. for one week. Sub-samples were taken throughout the incubation to quantify the monosaccharides generated from the conversion of cellulose and hemicellulose.

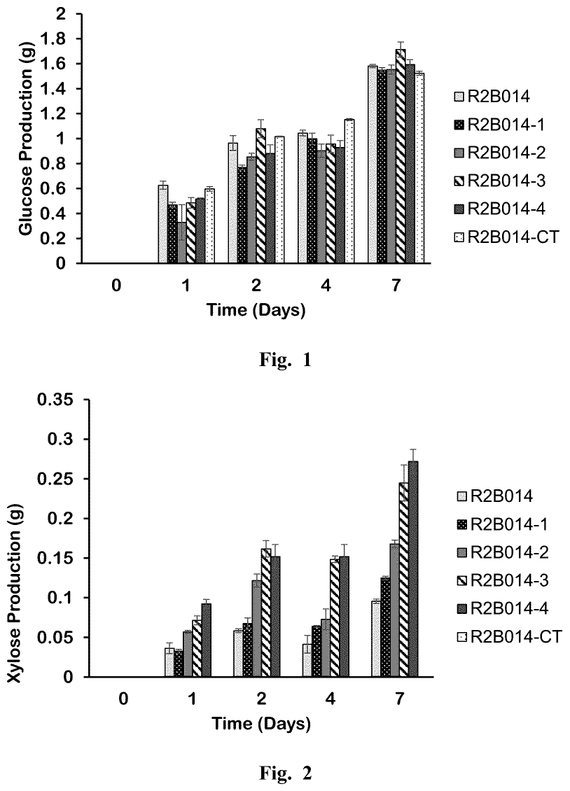

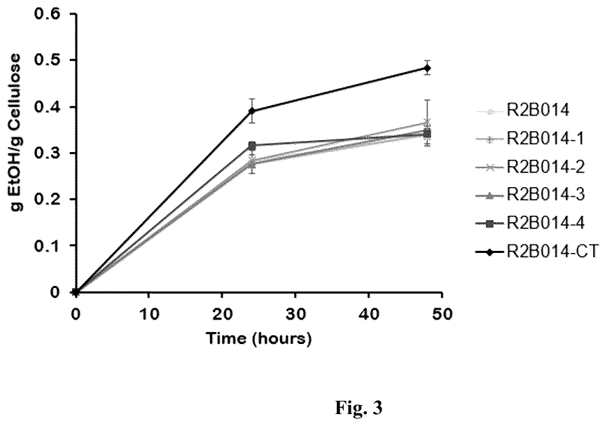

Production of glucose across all recycles of the substantially hemicellulose-free cellulose remains consistently similar throughout the saccharification reaction (FIG. 1). Xylose production, however, increases as the hemicellulose content of the cellulose batches increases (FIG. 2). As xylose accumulates in a bio-ethanol production system, this will affect the final yields of ethanol obtained, as inhibitors are formed and accumulated over time if the yeast in use cannot metabolize it. This experiment also demonstrates that a commercially available cellulase enzyme blend contains not only cellulase enzymes, but hemicellulase enzymes as well. It is therefore favourable and more efficient to remove the hemicellulose in order to obtain optimal bio-ethanol yields from a pure, delignified cellulose, without the need for genetic modification.

It is understood that lower hemicellulose and lower lignin content in cellulosic components used to produce ethanol will generate higher ethanol yields when using a non-genetically modified ethanologenic organism (such as Saccharomyces cerevisiae). The presence of lignin is detrimental to the conversion of cellulose to glucose as enzymes are known to adsorb on the surface of the lignin and consequently inhibit the enzymatic hydrolysis efficiency of the former. The presence of hemicellulose is also detrimental to the conversion of glucose to ethanol. Hemicellulose acts as a physical barrier which covers the cellulose and blocks the cellulase from performing enzymatic hydrolysis thereon. It is known to be preferably to remove hemicellulose from a cellulosic component allows the cellulase to gain better access to the cellulose and hydrolyze the latter.

### Experiment #2: Conversion to Ethanol

The conversion of the cellulosic component to ethanol for each one of the batches obtained through the single delignification treatment step with a modified Caro's acid yielded very good ethanol production as can be seen in FIG. 3. For the caustic treatment sample, the ethanol production was even more enhanced when hemicellulose was almost entirely removed (see FIG. 3). The delignified cellulosic component followed with a caustic treatment produced more ethanol compared to the single treatment step samples with less than 12.5% hemicellulose remaining. This substantiates the detrimental impact of the presence of hemicellulose on the final bioethanol yields.

Given this information, it is believed that idle ethanol plants located around the world could re-start operations of cellulose conversion to glucose (and subsequently, ethanol) using a biomass feedstock and treatment according to the above was employed rather than using corn, sugar cane or conventional kraft pulp. Moreover, the implementation of a method according to a preferred embodiment of the present invention would essentially “dovetail” with the delignification process of a lignocellulosic biomass by using a modified Caro's acid, and the production of ethanol with the cellulose obtained from the delignification process using lignocellulosic biomass. The person skilled in the art will recognize that by employing a cellulosic component obtained from a delignification approach using a modified Caro's acid as described herein, one can circumvent the need of any further or subsequent treatment step of said cellulosic component prior to using it for bioethanol production. As is also understood by the person skilled in the art, such additional treatment steps (to remove hemicellulose) is understood to likely be not economically viable in many, if not all countries, when the ultimate goal of the resulting cellulosic component is to be further converted for ethanol generation. It is also understood by a person skilled in the art that such a high cellulosic purity, low hemicellulose content pulp is beneficial for cellulosic ethanol processes as it minimizes the issues brought by the presence of hemicellulose in Kraft pulp processes and the like.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A cellulosic composition comprising cellulose and hemicellulose, said cellulosic component obtained from a delignification process using a modified Caro's acid selected from the group consisting of:
composition A; composition B and composition C;
wherein said composition A comprises:
sulfuric acid in an amount ranging from 20 to 70 wt % of the total weight of the composition;
a modifier component comprising an amine moiety and a sulfonic acid moiety selected from the group consisting of: taurine; taurine derivatives; and taurine-related compounds; and
a peroxide;
wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
sulfuric acid;
a two-part modifier component comprising:
a compound comprising an amine moiety;
a compound comprising a sulfonic acid moiety; and
a peroxide;
for the saccharification of cellulose into glucose and subsequent fermentation into ethanol, wherein said cellulosic component comprises at least 85 wt % of said cellulose and, at most, 15 wt % of said hemicellulose as a result of said delignification process.

2. A cellulosic composition comprising cellulose, hemicellulose and lignin obtained from the treatment of lignocellulosic biomass with a modified Caro's acid in the fermentation of cellulose into cellobiose and ethanol, where the cellulosic component is characterized in that its content of lignin is below 1 w/w % and hemicellulose is below 15 w/w % wherein said modified Caro's acid is selected from the group consisting of: composition A; composition B and composition C;
wherein said composition A comprises:
sulfuric acid in an amount ranging from 20 to 70 wt % of the total weight of the composition;
a modifier component comprising an amine moiety and a sulfonic acid moiety selected from the group consisting of: taurine; taurine derivatives; and taurine-related compounds; and
a peroxide;
wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
sulfuric acid;
a two-part modifier component comprising:
a compound comprising an amine moiety;
a compound comprising a sulfonic acid moiety; and
a peroxide.

3. The composition according to claim 1 where the cellulose to hemicellulose weight ratio in the cellulosic component is 6:1 or more.

4. The composition according to claim 1 where the cellulose to hemicellulose weight ratio in the cellulosic component is 7:1 or more.

5. The composition according to claim 1 where the cellulose to hemicellulose weight ratio in the cellulosic component is 10:1 or more.

6. The composition according to claim 1 where the cellulose to hemicellulose weight ratio in the cellulosic component is 12:1 or more.

7. A method of increasing an amount of ethanol produced from a fermentation unit comprising:
providing the composition of claim 1; and
operating the fermentation unit wherein the composition of claim 1 is used as an additive to organic material for bioethanol production.

8. The composition according to claim 1, wherein the cellulosic component comprises at least 85% cellulose.

9. The composition according to claim 1, wherein the substantially hemicellulose-free cellulosic component comprises at least 90% cellulose.

10. The composition according to claim 1, wherein the substantially hemicellulose-free cellulosic component comprises at least 92.5% cellulose.

11. A method of obtaining ethanol from a lignocellulosic biomass where said method comprises the following steps:
preparing a delignification mixture and delignifying a lignocellulosic biomass using a modified Caro's acid selected from the group consisting of: composition A; composition B and composition C;
wherein said composition A comprises:
sulfuric acid in an amount ranging from 20 to 70 wt % of the total weight of the composition;
a modifier component comprising an amine moiety and a sulfonic acid moiety selected from the group consisting of: taurine; taurine derivatives; and taurine-related compounds; and
a peroxide;
wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
sulfuric acid;
a two-part modifier component comprising:
a compound comprising an amine moiety;
a compound comprising a sulfonic acid moiety; and
a peroxide;
wherein said delignification mixture comprises said lignocellulosic biomass and said modified Caro's acid;
recovering a solid portion of said delignification reaction mixture, wherein said solid portion comprises a substantially hemicellulose-free cellulosic component which comprises at most, up to 15% w/w hemicellulose;
exposing said recovered solid portion of the resulting reaction mixture to an enzyme mix comprising at least one cellulase enzyme to create a saccharification or fermentation system which breaks down the cellulose into a saccharified composition, e.g., oligosaccharides or ferments said saccharified composition to ethanol; and
optionally, feeding the saccharified composition to an organism, such as yeast, capable of fermenting sugars into ethanol.

12. The method according to claim 11, further comprising: wherein after fermentation, the liquid portion from the fermentation system is distilled to recover said ethanol.

13. A method to increase an amount of ethanol produced from a fermentation system comprising:
providing a substantially hemicellulose-free cellulosic component, which comprises at most, up to 15% w/w hemicellulose;
providing the cellulosic component as an additive to organic material used for bioethanol production; and
operating the fermentation system to produce ethanol.

14. The method according to claim 13 wherein the substantially hemicellulose-free cellulosic component is a partial replacement for the organic material.

15. The method according to claim 11 wherein said substantially hemicellulose-free cellulosic component comprises less than 30% of the amount hemicellulose present prior to a lignocellulosic biomass being delignified.

16. The method according to claim 11 wherein said substantially hemicellulose-free cellulosic component comprises less than 20% of the amount hemicellulose present prior to a lignocellulosic biomass being delignified.

17. The method according to claim 11 wherein substantially hemicellulose-free cellulosic component comprises less than 8% of the amount hemicellulose present prior to a lignocellulosic biomass being delignified.

18. The method according to claim 11 wherein substantially hemicellulose-free cellulosic component comprises less than 4% of the amount hemicellulose present prior to a lignocellulosic biomass being delignified.

19. A process to hydrolyze cellulose into glucose, said process comprising the following steps:
providing a reaction vessel;
providing a source of cellulose into said reaction vessel; wherein said source of cellulose has a hemicellulose content of less than 30% of the original hemicellulose content of lignocellulosic biomass from which it originated;
providing a microbial inoculum into said reaction vessel;
exposing said inoculum to said source of cellulose in an aqueous medium of pH of about 8 at a temperature ranging from 30° C. to 40° C. for a period of time ranging from 1 to 42 days;
optionally, recovering the supernatant comprising glucose; and
exposing the glucose to a bacterium, a fungi, or yeast, or a combination thereof which ferments the glucose to ethanol.

20. A process to hydrolyze cellulose into glucose, said process comprising the following steps:
providing a reaction vessel;
providing a source of cellulose into said reaction vessel; wherein said source of cellulose has a hemicellulose content of less than 30% of the original hemicellulose content of lignocellulosic biomass which it originated from;
providing an enzyme blend into said reaction vessel;
exposing said enzyme blend to said source of cellulose in an aqueous medium of pH of about 4-6 at a temperature ranging from 40° C. to 55° C. for a period of time ranging from 1 to 7 days;
optionally, recovering the supernatant comprising glucose; and
exposing the glucose to a bacterium, a fungi, a yeast, or a combination thereof which ferments the glucose to ethanol.
